(19) United States
(12) Patent Application Publication
(10) Pub. No.: US 2005/0027164 A1
(43) Pub. Date: Feb. 3, 2005

(54) Vision catheter

(75) Inventors: Louis J. Barbato, Franklin, MA (US); Mark A. Hamm, Lynnfield, MA (US); Yem Chin, Burlington, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED Life Systems, Inc.

(21) Appl. No.: 10/793,482

(22) Filed: Mar. 4, 2004

Related U.S. Application Data
(63) Continuation-in-part of application No. 10/630,440, filed on Jul. 29, 2003.

(51) Int. Cl. A61B 1/00

(52) U.S. Cl. 600/101

(57) ABSTRACT

A catheter with a small optical fiber or bundle of fibers includes a scanning mechanism constructed with the use of any vibration capable component. Magnetic, piezoelectric or other mechanisms are used to vibrate the end of the fiber and thus create a scanning effect which extends the field of view. One or more lenses may be utilized, including a lens attached to the distal tip of the image fiber, or a lens attached to the distal tip of the catheter for creating an image plane which can be scanned by the fiber. In one embodiment, multiple light sources may be connected to the fiber for enabling the use of field sequential color techniques for real-time imaging, as well as real-time fluorescent imaging for disease detection. A photodetector assembly connected to the proximal end may contain both filtered and unfiltered detectors for use with both standard imaging and fluorescent imaging. The resulting vision catheter is relatively inexpensive and disposable.

### CROSS-REFERENCE TO RELATED APPLICATION

This application is a continuation-in-part of U.S. Patent Application Ser. No. 10/630,440, filed Jul. 29, 2003, priority from the filing date of which is hereby claimed under 35 U.S.C. § 120.

### FIELD OF THE INVENTION

The present invention relates to medical devices, and in particular to a catheter with imaging capabilities.

### BACKGROUND OF THE INVENTION

An endoscope is a type of catheter that has imaging capabilities so as to be able to provide images of an internal body cavity of a patient. Most minimally invasive surgical procedures performed in the GI tract or other internal body cavities are accomplished with the aid of an endoscope. A typical endoscope has an illumination channel and an imaging channel, both of which may be made of a bundle of optical fibers. The illumination channel is coupled to a light source to illuminate an internal body cavity of a patient, and the imaging channel transmits an image created by a lens at the distal end of the scope to a connected camera unit or display device.

As an alternative to an imaging channel made of a bundle of optical fibers, a semiconductor-type camera can also be attached onto the distal tip. One drawback of this alternative is that such cameras are relatively large in size, in comparison to the dimensions needed for certain surgical procedures. Another issue with either the semiconductor-type camera or the bundle of fibers, is that the ability to see a larger area requires moving the camera or the bundle of fibers. This type of movement is relatively complex to implement, and requires even more area. Furthermore, while endoscopes are a proven technology, they are relatively complex and expensive to manufacture.

Given these shortcomings, there is a need for a relatively small imaging device that is inexpensive and disposable.

### SUMMARY OF THE INVENTION

To address these and other concerns, the present invention is a catheter that includes an imaging channel. The imaging channel may include an optical fiber bundle or a single optical fiber with a distal end and a proximal end. The field of vision of the imaging channel is increased by vibrating the distal end. A number of compact and relatively inexpensive technologies can be used to vibrate the distal end, such as electric coils, piezoelectric crystals, and microelectrical mechanical systems (MEMS). Other types of energy that can be used include ultrasound or frequency modulation.

In an embodiment utilizing an electrical coil, a metal-type ring or object encases the distal end and is contained in a housing with the electrical coil for vibrating the distal end in a controlled manner. This produces a scanning effect in that as the distal end moves, the field of vision at the distal end effectively increases. In alternate embodiments, the housing may contain other technologies for creating the movement, such as piezoelectric crystals, MEMS, etc. An objective lens or a series of lenses is placed in front of the distal end to magnify the image. A focusing screw mechanism is incorporated so that the image can be focused. At the proximal end, an imaging device such as a CCD, CMOS, pin hole, or photo diode camera is positioned so as to capture and transfer the image to either a processor or a computer that is able to store or display the image. A light processing box is located between the camera and the proximal end, which provides the source for the light that illuminates the imaged area.

In accordance with another aspect of the invention, lenses may be utilized to further enhance the system. For example, a lens can be used on the tip of the fiber to reduce the cone angle of light that can be received by the fiber. In general, when the optical fiber is vibrated to create a raster or spiral scan, whether in single mode or multi mode, lenses generally increase the performance with respect to both the field of view and the resolution. In one embodiment, a gradient index (a.k.a. “GRIN”) broad lens is attached to the distal tip of the fiber so as to reduce the cone angle viewed by the fiber, thus increasing the effective resolution of the scanned image. In another embodiment, modifying the distal tip of the fiber by melting the glass to form various shapes similar to lens shapes can be utilized to affect the way that the fiber collects light. In another embodiment, rather than being attached to the fiber, a lens may be placed in front of the fiber (e.g., attached to the vision catheter), so as to create an image plane which can be scanned by the fiber. In another embodiment, an imaging type gradient index broad lens may be utilized. The objective lens can provide a wide angle or telescopic view and creates an image plane that can be scanned by the bare optical fiber, which is vibrated to create the raster or spiral scan. In general, the smaller the fiber core or channel through which the light is transmitted at the center of the optical fiber, the better the resolution of an image created by scanning the optical fiber over the image plane of the objective lens. Conventional types of lenses such as ball lenses, among others, can also be used on the tip of the fiber to reduce the cone angle of light that can be received by the fiber. Conventional imaging lenses such as aspheric lenses, among others, can also be used in the fixed configuration that is placed in front of the imaging fiber (e.g., attached to the tip of the catheter) to create the image plane that is to be scanned by the fiber.

In accordance with another aspect of the invention, multiple light sources can be connected to the scanning fiber by using a fiber splitter/combiner. This enables the use of field sequential color techniques for real-time imaging, as well as real-time fluorescent imaging for disease detection. In such an embodiment, the photodetector assembly connected to the proximal end may contain both filtered and unfiltered detectors for use with both standard imaging and fluorescent imaging.

In accordance with another aspect of the invention, a system that can steer the distal end of the fiber bundle or single fiber is utilized to steer or increase the field of view without moving the device. Whether an imaging lens is utilized on the tip of the bundle, or a fixed objective lens is used on the distal tip of the catheter or guidewire that creates the image plane to be scanned by the fiber bundle, the steering of the distal end of the bundle further increases the field of view.

It will be appreciated that the vision catheter of the present invention includes components that are widely available and that can easily be assembled. The simple design thus allows for the production of catheters that are relatively inexpensive and disposable and which have imaging capabilities while still remaining relatively small in diameter.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a vision catheter formed in accordance with one embodiment of the present invention;

FIG. 2 shows an imaging system including a vision catheter combined with a processor and monitor for displaying a sensed image;

FIG. 3 shows a lens attached to the distal tip of a fiber;

FIG. 4 shows a lens attached to the distal tip of the catheter for creating an image plane that is to be scanned by the distal tip of a fiber; and

FIG. 5 shows multiple light sources connected to a scanning fiber.

### DETAILED DESCRIPTION OF THE PREFERRED EMBODIMENT

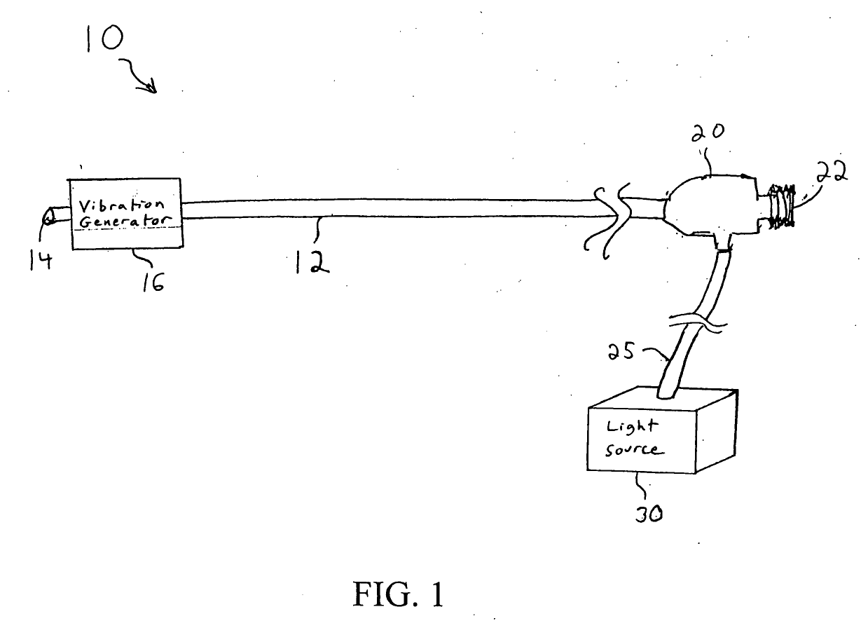

FIG. 1 is a diagram of a vision catheter 10 formed in accordance with the present invention. The vision catheter 10 includes a flexible imaging cable 12 having a polished distal end 14. In one embodiment, the flexible imaging cable 12 may include a group of standard clad optical fibers. In general, the optical fibers will include one or more imaging fibers and one or more illumination fibers. The imaging fibers transmit image information detected at the distal end 14 of the imaging cable 12. The illumination fibers are coupled to a light source so as to provide illumination at the distal end 14 of the imaging cable 12.

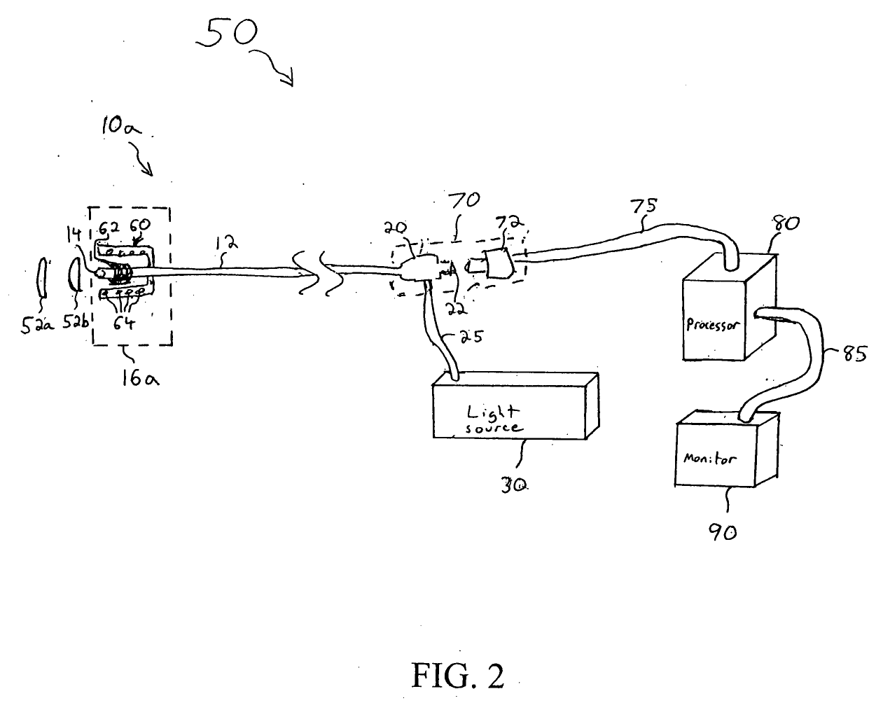

The vision catheter 10 also includes a vibration generator 16. In accordance with the present invention, the vibration generator 16 vibrates the distal end 14 of the imaging cable 12. This essentially produces a scanning effect in that as the distal end 14 moves, the field of view that is sensed by the distal end 14 effectively increases. As will be described in more detail below with reference to FIG. 2, the sensed image may be transferred to a computer or processor, and may further be recorded and/or displayed on a monitor.

The imaging cable 12 also includes a proximal end that is received within a housing 20. The housing 20 also includes a light splitter (not shown) which receives light through a cable 25 from a light source 30. The cable 25 may include a group of standard clad optical fibers that function as illumination fibers for carrying the light from the light source 30 to the light splitter within the housing 20. The light from the light splitter within the housing 20 is provided through the one or more illumination fibers in the imaging cable 12 to the distal end 14 of the imaging cable 12 for illuminating the imaged area. The housing 20 also includes an aperture 22 through which the image signals from the proximal end of the imaging cable 12 can be received.

FIG. 2 is a diagram of an imaging system 50 including a vision catheter 10a coupled to a processor 80 and a monitor 90. The vision catheter 10a includes a vibration generator 16a. The vibration generator 16a includes a metal ring 62 and electromagnetic coils 64. The metal ring 62 is placed around the imaging cable 12 at the distal end 14, and provides the mechanism for the coils 64 to vibrate the distal end 14 of the imaging cable 12 through the use of electromagnetic energy. In alternate embodiments, other technologies may be utilized in the vibration generator, such as piezoelectric crystals or microelectrical mechanical systems (MEMS). Further types of energy that can be used include ultrasound or frequency modulation.

A series of objective lenses 52a and 52b are placed in front of the imaging cable 12 to focus and magnify the image. A focusing mechanism such as a screw (not shown) may be incorporated so that the image sensed by the imaging cable can be better focused. A housing 70 includes the housing 20 which receives the proximal end of the imaging cable 12. The housing 70 also includes an imaging device 72 which is positioned relative to the aperture 22 so as to capture and transfer the image signals from the proximal end of the imaging cable 12. The imaging device 72 may be a CCD, CMOS, pin hole, photodiode camera, or other type camera. The imaging device 72 transfers the image through a cable 75 to a processor 80. The processor 80 may store or display the image. When the image is to be displayed, the processor may provide image signals through a cable 85 to a monitor 90.

As known in the art, a system may be provided for steering the distal end 14 of the flexible imaging cable 12, so as to steer or increase the field of view without otherwise moving the vision catheter 10. In general, whether an imaging lens is utilized on the tip of the distal end 14, or a fixed objective lens is attached to the distal tip of the vision catheter or guidewire so as to create an image plane to be scanned by the fiber bundle, the steering of the distal end 14 increases the field of view.

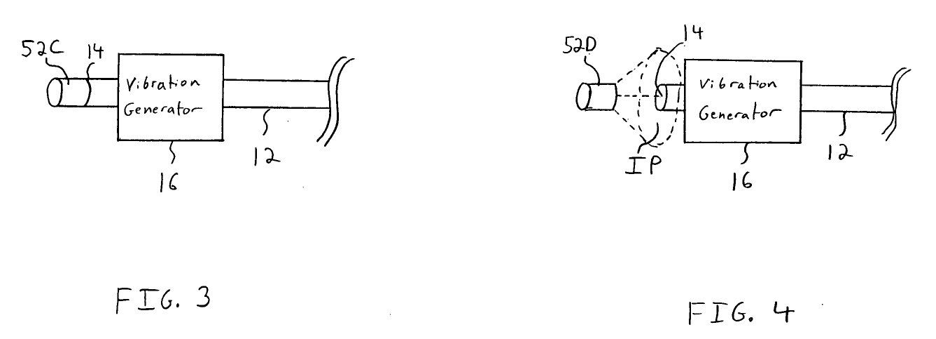

FIG. 3 is a diagram illustrating a lens attached to the distal end of a fiber. More specifically, similar to the vision catheter described above, FIG. 3 illustrates a flexible imaging cable 12 having a distal end 14. A vibration generator 16 vibrates the distal end 14 of the imaging cable 12. A lens 52C is attached to the distal end 14.

The lens 52C is useful in that in general when an optical fiber is vibrated to create a raster or spiral scan, whether single mode or multi mode, lenses may be utilized to increase the performance with respect to both the field of view and the resolution. In one embodiment, the lens 52C is a gradient index (a.k.a. “GRIN”) rod lens that can reduce the cone angle viewed by the fiber in the flexible imaging cable 12, thus increasing the effective resolution of the scanned image. A gradient index rod lens lends itself to this type of application because of its cylindrical shape. In other embodiments, other conventional types of lenses, such as ball lenses, can be used to reduce the cone angle of light that is received by the fiber. Since an optical fiber transmits light received from a cone angle related to its numerical aperture (NA), it is desirable in some embodiments to utilize either a lens attached to the distal tip of the fiber, or else utilizing a fixed objective lens located in front of the fiber (e.g., attached to the tip of the catheter). In another embodiment, the distal tip of the fiber may be modified by melting the glass at the distal tip to form various shapes similar to the lens shapes so as to alter the way that the fiber collects light.

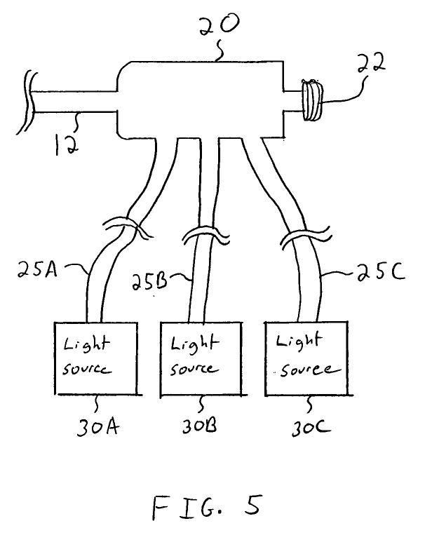

FIG. 4 illustrates a lens placed in front of the distal tip of a fiber for creating an image plane. More specifically, FIG. 4 shows a flexible imaging cable 12 having a distal end 14. A vibration generator 16 vibrates the distal end 14 of the imaging cable 12. A lens 52D is placed in front of the distal end 14 (e.g., fixedly attached to the distal tip of the catheter). The lens 52D is shown to create an image plane IP. In one embodiment, the lens 52D is a gradient index rod lens. In other embodiments, other conventional imaging lenses, such as aspheric lenses, can be used. The objective lens 52D provides a wide-angle or telescopic view and creates the image plane IP that can be scanned by the bare optical fiber in the flexible imaging cable 12. In this case, the smaller the fiber core, or channel through which light is transmitted at the center of the optical fiber, the better the resolution of an image created by scanning the optical fiber over the image plane IP of the objective lens 52D.

FIG. 5 is a diagram showing multiple light sources connected to the scanning fiber. More specifically, FIG. 5 shows an imaging cable 12 which includes a proximal end that is received within a housing 20. The housing 20 includes a fiber splitter/combiner (not shown) which receives light through cables 25A, 25B, and 25C, from light sources 30A, 30B, and 30C, respectively. The cables 25A, 25B, and 25C may include a group of standard clad optical fibers that function as illumination fibers for carrying the light from the light sources 30A, 30B, and 30C to the light splitter/combiner within the housing 20. The light from the light splitter/combiner within the housing 20 is provided through the one or more illumination fibers in the imaging cable 12 to the distal end 14 of the imaging cable 12 for illuminating the imaged area. The housing 20 also includes the aperture 22 through which the image signals from the proximal end of the imaging cable 12 can be received.

The multiple light sources 30A, 30B, and 30C are connected to the scanning fiber by utilizing the fiber splitter/combiner that is located within the housing 20. The use of multiple light sources enables the use of field sequential color techniques for real-time imaging, as well as real-time fluorescent imaging for disease detection. The photodetector assembly connected to the proximal end (as illustrated in FIG. 2) may contain, in the embodiment of FIG. 5, both filtered and unfiltered detectors for use with both standard imaging and fluorescent imaging.

It will be appreciated that the present invention provides a vision catheter that is relatively easy to build and which can be made from widely available components. Prior vision systems, such as endoscopes, tended to be relatively complex and expensive. The vision catheter of the present invention is relatively inexpensive and disposable.

While the preferred embodiment of the invention has been illustrated and described, it will be appreciated that various changes can be made therein without departing from the spirit and scope of the invention. For example, the imaging cable may incorporate the use of an optical single pixel or multi-fiber glass or plastic imaging bundle. The catheter construction could also include the optical bundle such that it is sandwiched or co-extruded and made to have any number of lumens. Extrusion technology can be used to provide any desired level of variable stiffness, torque, or articulation that is desired.

With regard to the illumination, while the casing at the proximal end of the imaging cable has generally been described as including a light splitter, it will be understood that any appropriate light directing mechanism may be utilized to focus light down to the tip at the distal end of the imaging cable so as to illuminate the imaged area. A light source itself could be replaced with a self-contained white light LED contained within the housing. The intensity of the light could be controlled by software or by a balancing control knob.

With regard to the field of view, focusing, and magnification, the lens or lenses at the distal end of the imaging fiber could be made to be adjustable so as to further increase the field of view or to allow for focus and additional magnification. The lens at the distal tip could be designed to have extra lumens for flushing so as to clean the surface. A focusing screw mechanism could be used to adjust the movement of the fiber for image sharpness and could be controlled by using any focusing technology known in the art. In addition, the vision catheter could be modified to include a mirror, either attached to the fiber or separated and appropriately positioned to allow for side viewing of images. By providing a side viewing port for the catheter, this would allow for a catheter with cutting wires to be observed during a surgical procedure.

Additional technologies that could be utilized for the vision catheter include infrared or ultrasound. It will be appreciated that these are just some of the various changes that could be made without departing from the spirit and scope of the invention. Accordingly, the embodiments of the invention, as set forth above, are intended to be illustrative, not limiting.

1. A vision catheter, comprising:
an image channel comprising one or more imaging fibers and a distal end and a proximal end;
a vibration generator for vibrating the distal end, and
a lens located in front of the distal end.

2. The vision catheter of claim 1, wherein the lens is attached to the distal end.

3. The vision catheter of claim 1, wherein the lens is attached to the vision catheter so as to create an image plane that may be scanned by the distal end when it is vibrated.

4. The vision catheter of claim 1, wherein the lens is a gradient index rod lens.

5. The vision catheter of claim 1, wherein the imaging channel comprises an imaging cable and the one or more imaging fibers are optical fibers.

6. The vision catheter of claim 1, wherein the vibration generator comprises a metal ring and one or more electromagnetic coils, the metal ring being placed around the one or more imaging fibers, the electromagnetic coils being driven by electrical energy so as to vibrate the metal ring.

7. The vision catheter of claim 1, further comprising one or more illumination fibers for illuminating the imaged area.

8. The vision catheter of claim 7, further comprising a light source coupled to a light splitter for providing light to the one or more illumination fibers.

9. The vision catheter of claim 7, further comprising a plurality of light sources for providing light to the one or more illumination fibers, the plurality of light sources being utilized to enable the use of field sequential color techniques.

10. A vision catheter, comprising:
an image channel comprising one or more imaging fibers, one or more illumination fibers, a distal end and a proximal end;
a vibration generator for vibrating the distal end; and
a plurality of light sources for providing light to the one or more illumination fibers.

11. The vision catheter of claim 10, wherein the plurality of light sources are utilized to enable the use of field sequential color techniques for real-time imaging, as well as real-time fluorescent imaging for disease detection.

12. The vision catheter of claim 10, wherein the proximal end outputs sensed image singals, and the vision catheter further comprises an imaging device for receiving the sensed image signals from the proximal end.

13. The vision catheter of claim 12, wherein the imaging device comprises a photodetector assembly that comprises filtered and unfiltered detectors for use with both standard imaging and fluorescent imaging.

14. An imaging system for use in surgical procedures, comprising:
an imaging channel comprising one or more fibers; and
a motion generator comprising first and second movement elements, the motion generator being operable to cause the first movement element to move relative to the second movement element, the first movement element being coupled to the one or more fibers.

15. The imaging system of claim 14, wherein the motion generator causes the first movement element to vibrate so as to create a scan by the one or more fibers.

16. The imaging system of claim 14, wherein the motion generator causes the first movement element to move such that the one or more fibers perform a spiral scan.

17. The imaging system of claim 14, wherein the motion generator causes the first movement element to move such that the one or more fibers perform a raster scan.

18. The imaging system of claim 14, wherein a lens is attached to the one or more fibers.

19. The imaging system of claim 14, further comprising a lens for creating an image plane that can be scanned by the one or more fibers as the motion generator moves the first movement element relative to the second movement element.

20. The imaging system of claim 14, further comprising a plurality of light sources coupled to the one or more fibers.
